(19) United States
(12) Patent Application Publication
(10) Pub. No.: US 2005/0239111 A1
(43) Pub. Date: Oct. 27, 2005

(54) Method for screening compounds using consensus selection and multiple descriptor sets

(75) Inventors: Albert M. Van Rhee, Durham, NC (US); Laura C. Van Zant, Cary, NC (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ICAGEN, Inc., Durham, NC (US)

(21) Appl. No.: 11/094,738

(22) Filed: Mar. 29, 2005

Related U.S. Application Data
(60) Provisional application No. 60/562,366, filed on Apr. 14, 2004.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

A method for screening compounds for biological activity is disclosed. A test library of compounds is selected. Then, a first analytical model is formed using a first recursive partitioning process. The first recursive partitioning process is performed on at least some of the compounds in the test library of compounds and uses a first descriptor set. Subsequent analytical models are formed using subsequent recursive partitioning processes using the digital computer and use multiple descriptor sets. A consensus compound set is determined using the first analytical model and one or more of the subsequent analytical models.

### CROSS REFERENCE TO RELATED APPLICATIONS

This application claims benefit under 35 U.S.C. 119(e) of U.S. Provisional Patent Application No. 60/562,366, filed Apr. 14, 2004, which is herein incorporated by reference in its entirety for all purposes.

### BACKGROUND OF THE INVENTION

In recent years, combinatorial chemistry coupled with high-throughput screening (HTS) has dramatically increased the number of compounds that are screened against many biological targets. Despite the resulting explosion of screening data for a given target, hit rates still tend to be quite low (typically much less than 1%). In the discovery of, for example, novel, small molecule modulators (inhibitors, activators, or otherwise) of ion channels, it would be desirable to improve hit rates beyond those obtained with historically, randomly or diversely chosen compound collections.

The application of cheminformatics to high-throughput screening (HTS) data requires the use of robust modeling methods. Robust analytical models must be able to accommodate false positive and false negative data, yet retain good explanatory and predictive power.

Recursive partitioning processes have been used to create analytical models. However, in some instances, analytical models formed using recursive partitioning suffer from high false positive rates, especially with sparse data sets such as HTS data.

While the role of molecular diversity and the influence of false positive data on interpretation of HTS screening results has been the subject of much speculation, most computational methods described to date utilize confirmed data from compound collections that tend to be poorly diverse. On the one hand, the level of diversity in a screening set can be highly controlled. On the other hand, HTS data by their nature are unconfirmed, and will contain some level of false positive and false negative data. It is therefore desirable to develop a method that is sufficiently robust to accommodate false positives and false negatives without compromising the utility of the models. One of the present inventors has previously introduced consensus selection by multiple recursive partitioning trees as a method to address this issue in U.S. patent application Ser. No. 10/754,484, filed on Jan. 9, 2004 (which is incorporated by reference in its entirety). Here the present inventors aim to further improve model performance by leveraging the power of independently computed metrics.

### SUMMARY OF THE INVENTION

In embodiments of the invention, consensus selection is used as a procedure to decrease the false positive rate of recursive partitioning-based models.

One embodiment of the invention is directed to a method for screening compounds for biological activity comprising: a) selecting a test library of compounds; b) forming a first analytical model using a first recursive partitioning process that uses a first descriptor set, a digital computer, and at least some of the compounds in the test library of compounds; c) forming a second analytical model using a second recursive partitioning process that uses a second descriptor set, the digital computer, and at least some of the compounds in the test library of compounds; and d) determining a consensus compound set using at least the first analytical model and the second analytical model, wherein the first descriptor set and the second descriptor set have different groups of descriptors.

Another embodiment of the invention is directed to a method comprising a) selecting a test library of compounds; b) forming a first analytical model using a first recursive partitioning process that uses a first descriptor set, a digital computer, and at least some of the compounds in the test library of compounds; c) forming a second analytical model using a second recursive partitioning process that uses a second descriptor set, the digital computer, and at least some of the compounds in the test library of compounds; d) forming a third analytical model using a third recursive partitioning process that uses a third descriptor set, the digital computer, and at least some of the compounds in the test library of compounds; and e) determining a consensus compound set using at least the first, second, and third analytical models, wherein the first, second, and third descriptor sets have different groups of descriptors.

Other embodiments of the invention are directed to computer readable media for performing such methods.

The present application refers to the use of first, second, third, etc. analytical models for purposes of illustration. It is understood that the use of these terms does not limit the invention to exactly two, three, four, etc. analytical models. Some embodiments may use two or more analytical models, while other embodiments could use tens or even thousands of analytical models in a consensus selection process.

These and other embodiments of the invention are described in further detail below.

### BRIEF DESCRIPTION OF THE DRAWINGS

FIG. 1 shows a flowchart illustrating a method according to an embodiment of the invention.

FIG. 2 shows a flowchart illustrating some steps used in forming a recursive partitioning tree.

FIG. 3 shows an example of a portion of a recursive partitioning tree.

FIG. 4 shows a Venn diagram illustrating a consensus region between two overlapping models. In this Venn diagram, each dashed circle represents a single model or a set of models. The area indicated by the solid line represents the consensus region between two overlapping models, i.e., both models agree on the importance of the indicated region.

FIG. 5 shows another Venn diagram illustrating a consensus region between three overlapping models. In this Venn diagram, each dashed circle represents a single model or a set of models. The area indicated by the solid line represents the consensus region between three overlapping models, i.e., all three models agree on the importance of the indicated region.

FIG. 6 shows another Venn diagram illustrating a majority rule region between three overlapping models. In this Venn diagram, each dashed circle represents a single model or a set of models. The area indicated by the solid line represents the majority rule region between three overlapping models, i.e., at least 2 of the 3 contributing models agree on the importance of the indicated region.

FIG. 7 shows hysteresis observed with increasing numbers of trees in a consensus model. The diagram shows hysteresis between the low-to-high (L2H; open symbols; the model with the lowest number of knots is taken first, and then models with increasing knot size are added) and high-to-low (H2L; closed symbols; the model with the highest number of knots is taken first, and then models with decreasing knot size are added) sequence of additions over the same set of individual trees (Factor Analysis descriptors).

FIG. 8 shows a table with the properties of individual recursion trees.

FIG. 9 is a table, which illustrates consensus selection using multiple recursion trees.

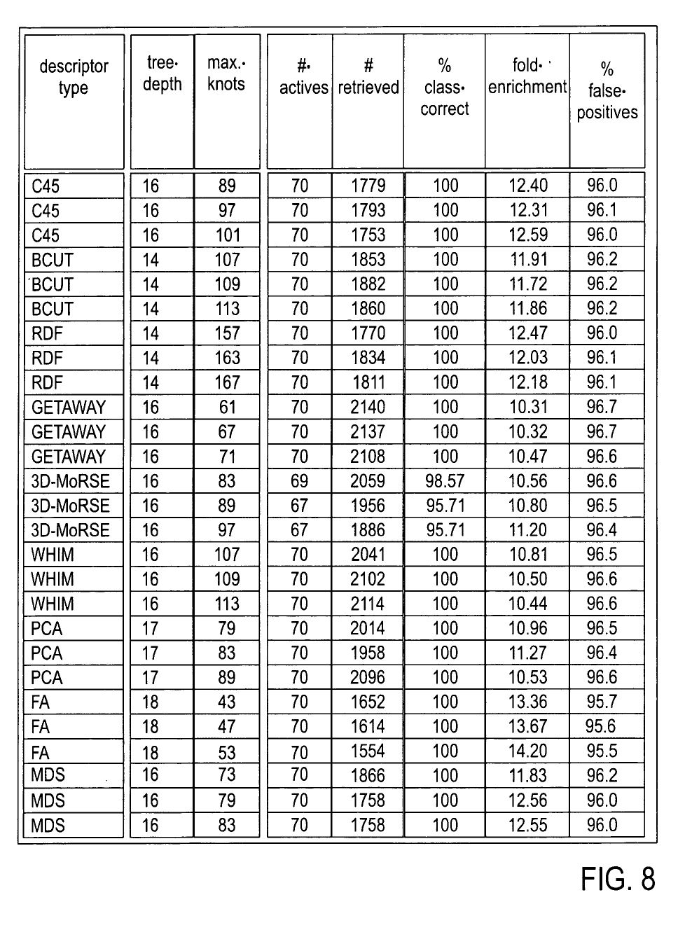

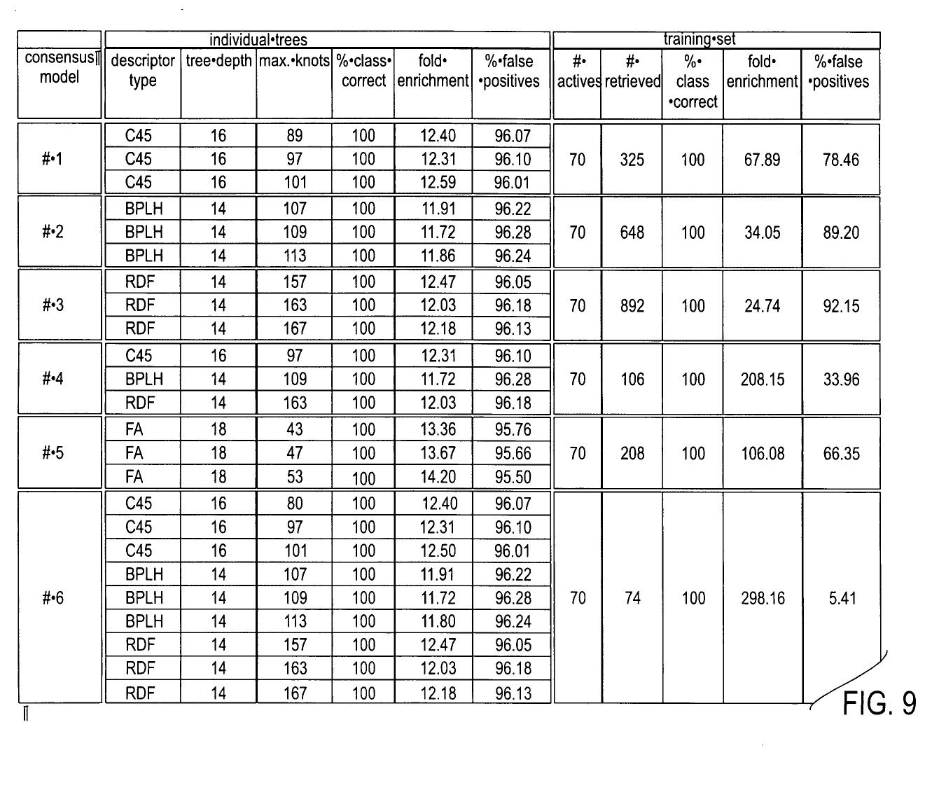

In FIGS. 8 and 9, C45 denotes Cerius² version 4.5 descriptors; BCUT denotes Diverse Solutions version 4.06 descriptors with explicit hydrogen atoms; RDF denotes Radial Distribution Function descriptors; RDF, GETAWAY, 3D-MoRSE, and WHIM are descriptors calculated using DRAGON PRO version 3.1; PCA denotes Principal Components Analysis; FA denotes Factor Analysis; and MDS denotes Multi-Dimensional Scaling.

### DETAILED DESCRIPTION

Embodiments of the invention leverage the power of independently derived metrics in the process of consensus selection by multiple recursion trees, thereby yielding unexpectedly high returns and predictive capabilities. In embodiments of the invention, independently derived metrics are used to describe the physicochemical properties of the chemical compounds under consideration.

A confounding factor in cheminformatics, and especially chemometric analyses, is the occurrence of colinearities and codependencies in a data matrix. The data matrix consists of a set of so-called independent or descriptive variables. Additionally, the data matrix contains one or more dependent or response variables. The response variables are usually obtained by experimental procedures, such as HTS or analytical chemical experiments, whereas the descriptive variables are usually computed from the chemical structure and composition of a set of compounds. Computed properties are frequently referred to as “metrics”.Colinearities and codependencies arise in the data matrix when a change in one variable causes a change in other variables, e.g., molecular weight and molecular volume are highly correlated. Less obvious is a (inverse) dependency of oral absorption on molecular weight. The occurrence of multiple colinearities and codependencies results in the availability in the data matrix of multiple tunable parameters that have a varying impact on the response variable, but cannot be independently assessed.

One advancement achieved herein arises from the use of independently derived metrics in the process of consensus selection by multiple recursion trees. In preferred embodiments, after careful elimination of duplicate metrics (the same variable occurring in more than one metric), a set of independent metrics can be obtained. A suite of recursion models is then developed. A consensus selection is made across independently derived recursion trees based on independent metrics.

Whereas a single recursion tree may explain 100% of the active training examples, it will also predict inactive training examples to be active resulting in false positive rates sometimes in excess of 90%. The resulting fold enrichment is therefore limited to perhaps 10-fold. The use of consensus selection reduces the false positive rate considerably, resulting in a fold enrichment of 20-100 fold (an 80-90% false positive rate). See U.S. patent application Ser. Nos. 60/442,449 filed on Jan. 24, 2003, and Ser. No. 10/754,484, filed on Jan. 9, 2004, which are herein incorporated by reference in their entirety. The use of metrics-leveraged consensus selection, as disclosed herein, further reduces the false positive rate. For example, as illustrated in the examples below, when three single trees of independent metrics were used, a fold enrichment of 208-fold could be obtained (i.e., a 30% false positive rate). Moreover, when consensus models were built using three sets of three trees each of independent metrics, a fold enrichment of 298-fold could be obtained (i.e., less than a 10% false positive rate). Thus, embodiments of the invention can provide for consensus compound sets that have false positive rates that are less than about 50 or even about 30 percent, while maintaining a fold enrichment of over 100-fold.

These results indicate that the use of metrics-leveraged consensus selection is not restricted to the area of drug development programs alone, but can also be applied in the prioritization of drug candidates for safety pharmacology screening such as hERG/Torsade-des-pointes, and other adverse drug events. It is expected that embodiments of the invention can also be applied to tune toxicological properties, metabolic liabilities, and therapeutic ratios associated with undesirable side effects (i.e., target selectivity). This represents a significant improvement over previous state-of-the-art processes in the following ways: 1. It retains the prospective nature of a data mining exercise in trying to identify desirable pharmacological and pharmaceutical properties outside of the training set; 2. It greatly reduces the number of experiments with an expected negative outcome; and 3. It enables the use of the methodology for the identification of undesirable pharmacological properties in that a) it can accurately identify a great majority (or all) of the undesirable drug candidates in the training set, and b) it only prioritizes a small set of drug candidates outside of the training set for further and focused testing of adverse drug events.

Embodiments of the invention are therefore expected to increase the fidelity and safety of the compound screening process, reduce operating costs and throughput requirements, shorten timelines, and increase the reliability of the process.

In embodiments of the invention, analytical models are formed. These analytical models include recursion trees. The recursion trees (or recursive partitioning trees) are formed using recursive partitioning processes.

Recursive partitioning is a method whereby a group of samples (e.g., compounds) is recursively split at a branch point into two statistically distinct nodes. The data matrix consists of columns for each of the descriptors, and rows for each of the samples of a training set. Each descriptor column is subjected to a process called splitting, in which a range for a descriptor is split into subranges. By systematically varying the splitting process, the statistical significance of each descriptor and its correlated range is determined. Branch points (or nodes) are identified by systematically evaluating the data matrix for the possibility to divide the matrix into statistically differentiated subsets based on their assigned category. The statistically most significant split then becomes a branch point in the recursive partitioning tree. Each subset in the matrix is subsequently analyzed for further significant differentiation. The process ends either when there are no more significant splits to be obtained, or when the minimum number of samples per node is reached. Once a recursive partitioning tree is formed, it may then be desirable to prune the tree to the appropriate tree depth as defined at the outset of the process. Additional details about screening processes using recursive partitioning can be found in U.S. patent application Ser. No. 60/270,365 filed Feb. 20, 2001, and U.S. patent application Ser. No. 10/077,358, filed Feb. 15, 2002. Both of these patent applications are herein incorporated by reference in their entirety.

There are several measures for determining the success of a recursive partitioning analysis. Some measures for determining success are as follows:

“hit rate” refers to the number of compounds that are shown to have biological activity within a predetermined activity range expressed as a percentage of the number of compounds in a set of compounds being analyzed. The pre-determined cut-off may be determined in any suitable manner. For example, the “hit rate” for a model formed using a training set of compounds may be the percent of compounds classified as “highly active” by the model. The “hit rate” for a training set of compounds as empirically determined may be the percent of compounds that are classified as being “highly active” after the compounds are tested, and are confirmed as being highly active. The bounds of “highly active” can be determined by one of ordinary skill in the art.

“fold enrichment” is the hit rate predicted by a model divided by the hit rate of an entire training set as empirically determined. A high sensitivity combined with a high specificity yields high fold enrichment. “sensitivity” and “specificity” are described in further detail below.

“% class correct” is a measure of the number of compounds correctly predicted to be within a class or a predetermined range of activity (e.g., “highly active”) as a percentage of the total number of compounds in the set known to be within the class or the predetermined activity range. In the case of the active category, it is referred to as “sensitivity”, where it is also known as “% recovery”.In the case of the inactive category, it is referred to as “specificity”.For ease of readability, “% class correct” refers to the % class correct for the active class, i.e., the selectivity.

“% overall correct” is the total number of compounds, regardless of class, correctly classified by the model, i.e., the sum of all true positive and true negative assignments, expressed as a percentage of the entire training set. It is also known as “concordance”.

It is relatively easy to obtain a high concordance by simply classifying all compounds as inactive (99.75% overall correct), or to obtain a high sensitivity by classifying all compounds as active (100% class correct), but it is much harder to obtain a high sensitivity and fold enrichment while maintaining a high concordance. The false positive rate (i.e., the percentage of compounds identified by the model as having a high probability of being active, but not actually having demonstrable activity) and false negative rate (i.e., the percentage of compounds identified by the model as having a low probability of being active, but actually having demonstrated significant activity) are better indicators of overall model quality. Whereas it is virtually impossible to evaluate the false negative rate of any model without experimentally testing all possible compounds repeatedly, it is feasible to evaluate the impact of model parameters on the model's false positive rate. Statistical models typically try to minimize the total error expressed in the concordance, i.e. both the number of false negatives and false positives, but with highly imbalanced data sets such as HTS data this objective needs to be implicitly adjusted in the model parameters to avoid classifying all samples as inactive (see above).

Fold enrichment and % class correct are not independent. Rather, they are interdependent. As the models become more sophisticated, e.g., increased tree depth, the activity is more narrowly defined, and as a result, more false positives are eliminated from the model. However, the method concurrently also tends to eliminate more false negatives, i.e., a higher false negative rate, resulting in a better fold enrichment in the remaining models, but a lower overall % class correct.

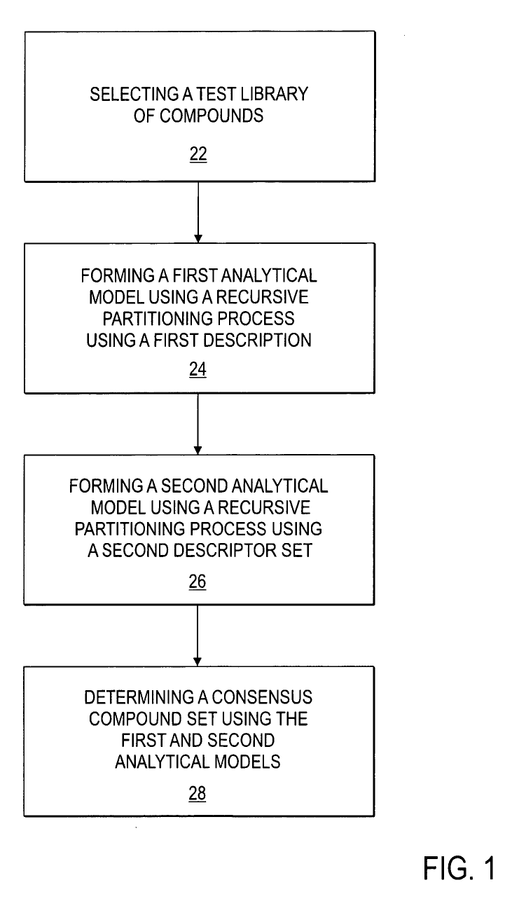

FIG. 1 shows a flowchart illustrating a method according to an embodiment of the invention. In the method, a test library of compounds is selected (step 22). After a test library of compounds is selected, a first analytical model is formed using a first recursive partitioning process using a digital computer (step 24). The first recursive partitioning process is performed on at least some of the compounds in the test library of compounds and uses a first descriptor set. For example, the first recursive partitioning process may use a training set of compounds, a first descriptor set “A”,and a digital computer. Concurrently with, or after performing the first recursive partitioning process, a second analytical model is formed using a second recursive partitioning process (step 26). The second recursive partitioning process is performed on at least some of the compounds in the test library, and uses a second descriptor set “B”,and uses the digital computer. The compounds used to form the second analytical model may be the previously mentioned training set of compounds or another set of compounds from the test library. References to “first” and “second” are intended to be non-limiting. Embodiments of the invention may, of course, create more analytical models and may use more recursive partitioning processes than two.

The first and second descriptor sets “A” and “B” comprise different groups of descriptors. In some embodiments, the different groups of descriptors may be partially or entirely different and may be partially or wholly independently derived. For example, a first descriptor set “A” may comprise a descriptor such as a AlogP, while a second descriptor set “B” may comprise a descriptor such as molecular weight. The first descriptor set “A” would not have the descriptor molecular weight and the second descriptor set would not have the descriptor AlogP. It is possible that some descriptors in the different descriptor sets may be similar, but at least 50% of the descriptors in the different descriptor sets are different and are independently derived. This is compared to the situation where multiple recursive partitioning trees are formed using the same descriptor set (e.g., two or more trees are formed using, for example, only BCUT descriptors) where all models share 100% commonality between the descriptors.

The first and second analytical models may respectively be two or more different recursive partitioning trees. The first and second analytical models may be, respectively, first and second recursive partitioning trees that are formed using the same or different set of compounds. The first and second recursive partitioning processes may be the same or different. For example, in some embodiments, the first and second analytical models may be formed using respectively different sets of parameters (e.g., tree depth, maximum knots, minimum number of samples per node, etc.), but may use the same training set of compounds. In another example, the parameters used to form the first and second analytical models may be the same (e.g., the same tree depth, maximum knots, and minimum number of samples per node), but the set of compounds used to form the first and second analytical models may be different. In these instances, different recursive partitioning trees are formed and these can be used to form a consensus model, which can be used to identify a consensus set of compounds.

A consensus compound set is then determined using the first analytical model and the second analytical model (step 28). As explained in further detail below, the Boolean intersection of two or more models can be used to identify the consensus compound set. Although the use of two analytical models is discussed for purposes of illustration, it is understood that more than two models can be used to form the consensus set of compounds.

I. Selecting a Test Library of Compounds

For each analytical model, a test library of compounds may be identified. In some embodiments, the test library has a high information content (i.e., it can be maximally diverse within the relevant pharmaceutical and/or therapeutic diversity space). The test library may contain any suitable type of compound and any suitable information that is related to the compounds. For example, the compounds in the test library may be chemical compounds or biological compounds such as polypeptides. The test library may contain data relating to the compounds in the test library. For example, each compound in the test library may have chemical data such as a hydrophobic index and a molecular weight associated with it. The tangible aspects of the test library, i.e., the physical compounds may be stored under appropriate chemical and biological safeguards (e.g. refrigeration) in appropriate media (e.g. dimethyl sulfoxide or phosphate-buffered saline solution) and containers (e.g. vials or multiwell plates), whereas the intangible aspects of the test library, i.e., the information related to the compounds may be stored in a database (e.g. ISIS™ and Oracle™).

The compounds in the test library may be obtained in any suitable manner. For example, the compounds in the test library may be selected from a pre-existing set of compounds. Alternatively or additionally, the compound library may contain compounds that have been created in a synthetic process such as a combinatorial synthesis process. The test library of compounds may be synthesized either by solid or by liquid phase methods known in the art. Additionally, compounds may be synthesized either individually, or by parallel methods known in the art. The synthetic process can be directed by synthetic feasibility without prior knowledge of the biological target. Additionally, compounds may only exist in a virtual sense (i.e. in an electronic form stored on a hard drive or in memory in a computer), such that the compounds' characteristics can be calculated and/or predicted without the compounds being physically present. Selected candidate (second or third tier) molecules can then undergo actual synthesis and testing.

Illustratively, a new compound data set consisting of 15,000 compounds can be created using, for example, parallel synthesis. The new compound data set can be compared to a pre-existing data set stored in a database such as an Oracle™ relational database management system. The relational database management system may store numeric data, alphanumeric data, binary data (such as in e.g., image files), chemical data, biological activity data, analytical models, etc. Members of the new compound data set that are not redundant of the pre-existing compound data set can then be retained and added to the database containing the pre-existing compound data set. The compound data set thus defined forms the testing library.

A commercial software package such as ISIS™ (Integrated Scientific Information System - a commercially available client/server application from MDL™ Information Systems, Inc., San Leandro, Calif.) can be used to compare data sets. ISIS™ can interface with, e.g., an Oracle™ database to allow for the searching of, for example, chemical data and structures stored in the Oracle™ database. ISIS™ allows a user to compare two compound data sets and determine the overlap (redundancy) between the data sets. Moreover, it allows the registration of redundant non-structure related data into the database while retaining only unique structure information. Of course, in other embodiments, data sets of compounds need not be compared to form a test set. For example, a number of compounds can be formed by a combinatorial synthesis process and then may be characterized. The compounds may form a test set without comparing the newly formed compounds with a pre-existing compound data set.

After forming the test library, some or all of the members of the compounds in the test library may be evaluated according to a predetermined pharmaceutical or a therapeutic profile. The evaluation can be conducted using, for example, Sybyl™, a commercially available molecular modeling suite of programs from Tripos, Inc., St. Louis, Mo. Using Sybyl™, 2D structural information can be transformed into 3D coordinates, and physicochemical properties based on either 2D or 3D chemical information can be obtained. 2D or 3D information can be used to determine if a compound is to be assigned a particular pharmaceutical or therapeutic profile. Using the pharmaceutical or therapeutic profile, only those compounds that fit the profile may be selected, and compounds that do not fit the profile are excluded, thus reducing the number of potential candidates. The selection of compounds using the pharmaceutical or therapeutic profile can take place before or after the analytical model is formed.

A typical pharmaceutical profile includes characteristics that make a compound desirable as a pharmaceutical agent. For example, one characteristic of a pharmaceutical profile may be the ability of a compound to dissolve in a liquid. If a compound dissolves in such liquid, then the compound fits the pharmaceutical profile. If it does not, then it does not fit the pharmaceutical profile. A typical therapeutic profile includes characteristics that make a compound desirable for a particular therapeutic purposes. For example, if the particular therapeutic purpose is to provide therapy to the brain, then the compound may have characteristics (e.g., small size) that permit it to pass the blood-brain barrier in a person. If the compound has these characteristics, then it fits the therapeutic profile. Characteristics relating to the pharmaceutical or therapeutic profile may be present in the test library and may be stored in a database along with each of the compounds in the test library. At any point, the profile information may be used to select compounds that have a higher likelihood of exhibiting a predetermined biological activity and/or are suitable for the particular pharmaceutical or therapeutic goal in mind.

A. Test Set and Training Set Selection from the Library of Compounds

A test set of compounds and a training set of compounds are selected from the test library of compounds. Typically, the number of compounds in the training set is less than 20% of the number of compounds in the test set. After the training set is formed, the test set may be the remaining compounds in the test library. For example, a test library may contain 700,000 molecules and the formed training set may consist of 15,000 molecules. The test set may then consist of the remaining 685,000 molecules.

The information content of the training set, whether a combinatorial library candidate for HTS or a statistical analysis data set, influences the efficiency and/or utility of the analysis methodology. For this reason different experimental design strategies have been developed for diverse compound selection from a larger chemical library or chemical diversity space. (Hassan, M. et al., Mol. Diversity, 2:64-74 (1996); Higgs, R. E. et al., J. Chem. Inf. Comput. Sci., 37:861-870 (1997).

In some embodiments, a diverse selection (DS) process can be performed using a D-optimal design strategy (Euclidian distance metric, Mean/Variance scaling, 75,000 Monte Carlo Steps at 300 K, Monte Carlo Seed of 12,379, termination after 1,000 idle steps, Gaussian alpha of 1.0, bucket size of 21 for the K-d tree, and taking the nearest 7 neighbors into consideration), as implemented in Cerius²™ (version 4.6; Accelrys Inc., San Diego, Calif.). In a DS process, compounds are selected to maximize representation in the test library. For example, if the compounds have characteristics that make them cluster in some way (e.g., by similar morphology), then fewer compounds in the cluster are selected in order to increase the representation of other compounds in the training set.

In other embodiments, a diverse selection of 5,000 compounds can be randomized with regard to the biological activity, yielding a diverse/randomized (DR) training set. The compounds in the diverse/randomized (DR) training set are randomly assigned biological activities, and a model is created. If the created model does not perform well, then the selected training set is desirable since the biological activities were randomly assigned and were not derived from actual testing. For example, 10 independent rounds of randomization can be performed where compounds are randomly (using a random number generator) assigned to the activity bins proportionately to their initial distribution, but without regard to their chemical structure and their measured biological activity.

In other embodiments, a random (RS) selection process can be used to form the training set. A training set formed by a random selection process is a stochastic sampling of a complete library, and therefore represents the information content in proportion to its distribution in the test library. In a sense, the information content is lower in a training set formed by random selection than by diverse selection. In a random selection process, densely populated areas with repetitive information are sampled more frequently than sparsely populated areas containing unique information.

II. Assaying

The compounds in the training set may be assayed to determine their biological activity. In some embodiments, an ion channel assay may constitute a homomultimeric, or heteromultimeric isoform of a single ion channel, or multiple ion channels related through their gene sequence (i.e., a “gene family”). If an assay constituting a homomultimeric or heteromultimeric ion channel of the same gene family is used, it is possible to establish a “gene family library space” by intersecting the screening results for different ion channel types (i.e., intersecting models). A “gene family library space” refers to a library consisting of compounds that work against more than one type of ion channel. For example, compounds in a gene family library space may work against two or more types of ion channels. A “gene specific library space” may be formed by subtracting the results of different screening results for different ion channel types (i.e., differentiating models). A “gene specific library space” refers to a library consisting of compounds that work preferentially against one type of ion channel.

Ion channels are membrane embedded proteins of multimeric composition with intrinsic ion conduction properties. The intended pharmacological endpoint, i.e. activation, prolongation of activation, modulation of the frequency or amplitude of activation, termination of activation, or block of the target ion channel, is dependent on the site and mode of binding of the ligand to the channel. The limitation of most Quantitative Structure-Activity Relationship (QSAR) methods is that a single (quasi-) linear equation is presumed to account for all biological activity, which is presumed to reside in a single binding site. Whereas this may hold true for selective, reversible, and competitive binding models, these conditions need not necessarily apply to HTS data sets. Furthermore, past research here and elsewhere indicates that it is very likely that many chemical modulators of ion channels, especially those that are endogenously regulated by membrane potentials (e.g., the Kᵥ gene family) or ion concentrations (e.g., Ca²⁺-sensitive channels), are noncompetitive, or uncompetitive, allosteric modulators. The problem can be addressed using Probabilistic Structure-Activity Relationship (PSAR) models based on Recursive Partitioning.

The biological activities determined by the assaying process may be defined by two or more classes (e.g., high activity and low activity). Preferably, the biological activities may be defined by three or more related classes (e.g., high activity, moderate activity, and low activity). For example, the screening assay determines the biological activity of each compound. Each compound is then assigned to a particular class with a predetermined activity range, based on the determined biological activity. In some embodiments, the activity ranges for the different classes may include “high activity”, “moderate activity”,“low activity”,and “inactive.” The skilled artisan can determine the quantitative bounds of the classes.

Any suitable assay known in the art may be used to determine the biological activity of the compounds in the test library. For example, the biological activity of the compounds may be determined using a high-throughput whole cell-based assay.

In preferred embodiments, the assay determines the ability of the compounds in the test set to modulate the activity of ion channels and the degree of activity. For example, the activity of an ion channel can be assessed using a variety of in vitro and in vivo assays, e.g., measuring current, measuring membrane potential, measuring ligand binding, measuring ion flux, (e.g., potassium, or rubidium), measuring ion concentration, measuring second messengers and transcription levels, using potassium-dependent yeast growth assays, and using, e.g., voltage-sensitive dyes, ion-concentration sensitive dyes such as potassium sensitive dyes, radioactive tracers, and electrophysiology. In a specific example, changes in ion flux may be assessed by determining changes in polarization (i.e., electrical potential) of the cell or membrane expressing the ion channel. A preferred means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., Pflügers. Archiv. 391:85 (1981).

In an illustrative assay for a potassium channel, samples that are treated with potential potassium channel modulators are compared to control samples without the potential modulators, to examine the extent of modulation. Control samples (untreated with activators or inhibitors) are assigned a relative potassium channel activity value of 100. Modulation is achieved when the potassium channel activity value relative to the control is distinguishable from the control. The degree of activity relative to the control is generally defined in terms of the number of standard deviations from the mean. For instance, if the mean is 0%, and the standard deviation is 25%, then the activity ranges could be defined as 1) 0-25%, i.e. within 1 standard deviation of the mean, 2) 25-50%, i.e. within 2 standard deviations from the mean, 3) 50-75%, i.e. within 3 standard deviations from the mean, and 4) 75-100%, i.e. within 4 standard deviations from the mean. These ranges of activity may correspond to, for example, inactive, weakly active, moderately active, and highly active, respectively.

III. Forming First, Second, Third and Subsequent Analytical Models

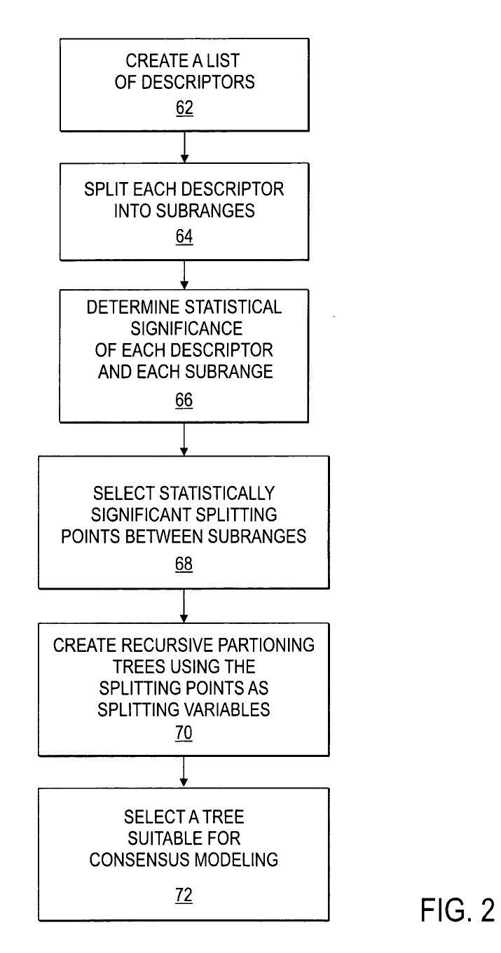

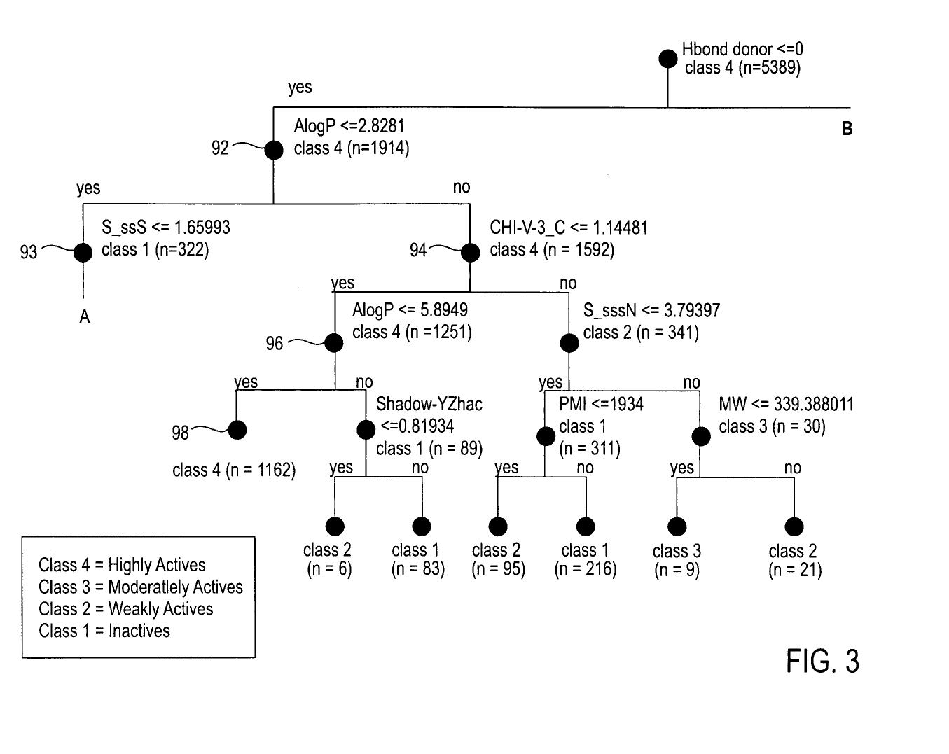

In one embodiment of the invention, two or more recursive partitioning trees may be formed from at least some of the compounds in the test library. The same or different sets of compounds may be used to form the different recursive partitioning trees. If the same sets of compounds are used, then the parameters used to form the trees may differ in some way. For example, the tree depth and/or the minimum samples per node may be varied to produce different recursive partitioning trees using the same set of compounds. Alternatively, different sets of compounds from a test library may be used to form respectively different recursive partitioning trees. Exemplary processes for forming recursive partitioning trees can be described with reference to FIGS. 2 and 3. As noted above, at least two trees that are to be used in the consensus selection process are formed using different descriptor sets.

Referring to FIG. 2, a list of descriptors is created to form a descriptor space (step 62). A descriptor may be binary in nature, i.e., it can denote the presence or absence of a feature but not its extent. For example, a descriptor named “heterocyclic” may denote the presence (1) or absence (0) of heteroatoms in a ring otherwise constituted by carbon atoms, but holds no information as to the number of heteroatoms present. Alternatively, a descriptor could be a continuous range descriptor. That is, it can denote the extent to which a particular feature is represented. For example, the molecular weight of a compound may be considered a continuous range descriptor. All molecules have a molecular weight, but the extent of the descriptor (e.g., a molecular weight as expressed in a range of Daltons) can be used to discriminate one molecule from another. Other examples of descriptors include the principal moment of inertia in a molecule's primary X-axis (PMI_X), a partial positive surface area (JURS_PPSA_{—}1), molecular density (Density), molecular flexibility index (phi), etc. In embodiments of the invention, hundreds or thousands of such descriptors can be considered when forming an analytical model.

A number of exemplary descriptors are provided in Cerius²™, commercially available from Accelrys, Inc., San Diego, Calif. Cerius²™ is capable of generating descriptors such as spatial descriptors, structural descriptors, etc. for evaluation. It is also capable of creating recursive partitioning trees. It also allows for the variation of variables such as knot limit, tree depth, and splitting method. In embodiments of the invention, the tree depths of the recursive partitioning trees created are systematically varied until the optimal tree(s) are determined.

Each descriptor is subjected to a process called splitting, in which the range (highest descriptor value minus lowest descriptor value) is split into subranges (step 64). By systematically varying the splitting process, the statistical significance of each descriptor and its correlated range is determined (step 66). Splitting points are identified by systematically evaluating the subranges for the possibility to divide the compounds into statistically differentiated subsets based on their assigned category (step 68). The statistically most significant splitting point then becomes a splitting variable in the recursive partitioning tree.

Illustratively, a descriptor such as molecular weight can be optimized. Based on past experience or knowledge, it may be determined that the molecular weight of the particular modulator being sought would have a molecular weight ranging from 23 to 20,000. The range of 23-20,000 can then be split into progressively smaller subranges. The training set data are then applied to these splits to determine which subrange is the optimal range. For example, if it is discovered that out of 200 candidate compounds, 50 compounds having a molecular weight between 23-10,000 exhibit high activity and 150 compounds having a molecular weight between 10,000 and 20,000 exhibit low activity, then the range of 23-10,000 is selected as the more preferred range. Since a molecular weight of 10,000 splits the data, it is a splitting point and may be referred to as a “knot”.“Splitting points” and “knots” are used interchangeably and refer to values that are used to split a range for a descriptor. The 23-10,000 molecular weight continuous range descriptor is then used as a splitting variable at a node in a classification and regression tree. For example, the variable MW (molecular weight) could be used in two consecutive splits: MW <=10,000 and MW >23, to define the preferred range of 23-10,000 used to classify compounds in the test set. In this example, only one descriptor with two knots is described for simplicity of illustration. However, in other embodiments, the number of knots per descriptor may be 1 to 100 or more. Narrow or broad ranges for the descriptors can be evaluated for statistical significance.

A. Forming Trees

A plurality of recursive partitioning trees is created (step 70). Tens or hundreds of trees may be generated in some embodiments. Each tree uses the descriptors, as calculated and optimized above, as splitting variables to form splits in the data. Many such trees are created while varying such parameters as the knot limit, tree depth, and splitting method. Then, one or more trees are selected (step 72) as an analytical model(s). The tree that is selected is the one that differentiates the data the best according to biological activity and/or is one that is best suited for the subsequent consensus selection process. This is described in further detail below. The same general process may be repeated to form a second, third, and subsequent analytical model.

As noted above, each tree is formed using a different descriptor set. The respective descriptor sets may have respectively different groups of descriptors. For example, different trees or sets of trees may be formed using the following distinct descriptor sets, which are described in further detail above and below: C45, BCUT, RDF, GETAWAY, 3D-MoRSE, and WHIM descriptors. Of course, the process described in this application is not limited to these particular sets of descriptors. At least some (e.g., more than 50%) of the descriptors in the different descriptor sets are independently derived.

In a typical recursive partitioning tree, parent nodes are split into two child nodes. A splitting variable splits the training set compounds into two statistically significant groups, and these two groups are classified into two respective child nodes. A Student's t-test may be used to determine the statistical significance of the split. In forming a tree, splitting methods such as the Gini Impurity, Twoing Rule, or the Greedy Improvement can be used to split the compounds. These methods are well known in the art and need not be described in further detail here.

Once a best split is found, the classification and regression tree process repeats the search process for each child node, continuing recursively until further splitting is impossible or stopped. Splitting is impossible if only one case remains in a particular node or if all the cases in that node are of the same type. Alternatively, the process ends when there are either no more significant splits to be obtained, or when the minimum number of compounds per node is reached. The nodes at the bottom of a tree (i.e., where further splitting stops) are called terminal nodes. Once a terminal node is found, the node is classified. The nodes can be classified by, for example, a plurality rule (i. e., the group with the greatest representation determines the class assignment), or the nodes can be classified by a weighted means rule (i.e., the compounds with the highest weighted values contribute disproportionately more to the mean value of the node than those with the lesser weighted values). The tree may be pruned to the appropriate tree depth as defined at the outset of the process.

FIG. 3 shows an example of a portion of a recursive partitioning tree. The area where the letters “A” and “B” are present would have additional nodes, branches, etc. For purposes of clarity, these additional tree structures have been omitted. In this example, a node 92 may be characterized as a highly active node where the tree initially classifies 1914 members of a test set as being highly active. Then, the splitting variable “AlogP <=2.8281”may be applied to the 1914 compounds at the node 94. “AlogP” is a property of a chemical compound that is described in greater detail in Ghose A. K. and Crippen G. M. (J. Comput. Chem., 7, 1986, 565). Compounds that satisfy this condition are placed in node 93 while compounds that do not are placed in node 94. The compounds assigned to these nodes 93, 94 are further split in a similar fashion, but with different rules. The classification of each node 93, 94 can be determined by determining which particular activity (i.e., highly active, moderately active, weakly active, or inactive) predominates at the node. The compounds can be split until a terminal node 98 is reached. In some embodiments, the terminal node may contain compounds, all of which (or a majority of) have the same biological activity. The terminal node may then be characterized by the determined biological activity. In this particular example, the nodes 92, 94, 96, 98 are all characterized as highly active nodes. The compounds classified in terminal node 98 satisfy the following conditions:
Hbond donor [symbol] 0, yes (“Hbond donor” is the number of hydrogen bond donors)
AlogP [symbol] 2.8281, no (“AlogP” is a calculated octanol/water partitioning coefficient)
CHI-V-3_C [symbol] 1.14481, yes (“CHI-V-3_C” is a 3rd Order Cluster Vertex Subgraph Count Index)
AlogP [symbol] 5.8949, yes (“AlogP” is a calculated octanol/water partitioning coefficient)

This set of rules or descriptors can be used to select a class of compounds that are expected to have a “high biological activity”.In this example, the 1162 compounds in the terminal node 98 may serve as potential candidates for modulators. If desired, these compounds may be analyzed (e.g., by a computer or the skilled artisan) to determine if there are any chemotypes that are prevalent in the terminal node compounds. These chemotypes may serve as a basis for further research or analysis. Advantageously, in embodiments of the invention, potentially effective chemotypes can be identified in addition to providing enhanced hit rates.

IV. Consensus Selection & Majority Rule Election

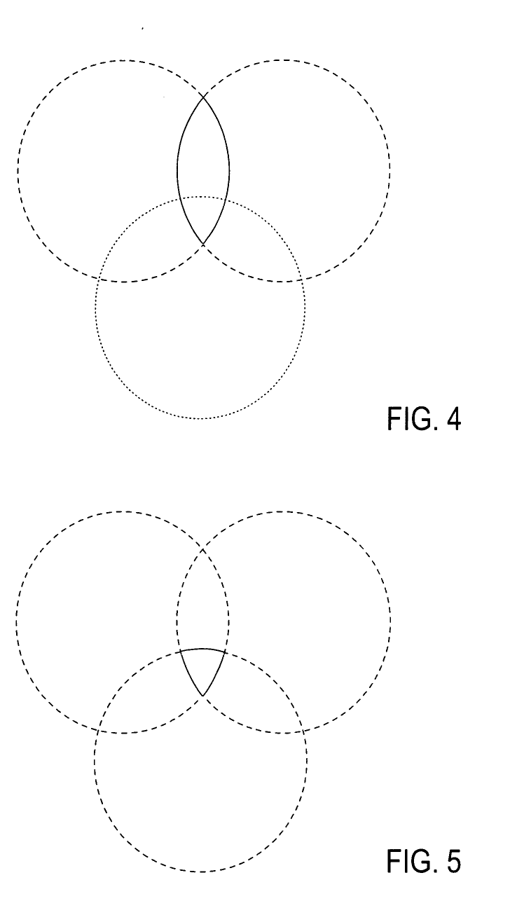

Ensemble methods, i.e., those methods that depend on more than one model, rely on a set of simple Boolean instructions. In its simplest form, it is either the union (FIG. 4, dashed black lines), or the intersection (FIG. 4, solid black lines) of two mathematical sets.

“Consensus selection” is a process for group decision-making. It is a method by which a group of models agrees on a narrowly defined set of particular solutions. The input and statistics of all participating models are gathered and synthesized to arrive at a final model satisfying the conditions of each of the contributing models. The consensus selection process involves the determination of the Boolean intersection of a set of models (at least 2, in theory unlimited, individually derived models), thereby emphasizing the probabilities of the consensus set (FIG. 5, solid lines), and de-emphasizing the probabilities of the contributors for each of the models excluded from the consensus set, i.e., the dissenting sets (FIG. 5, dashed lines).

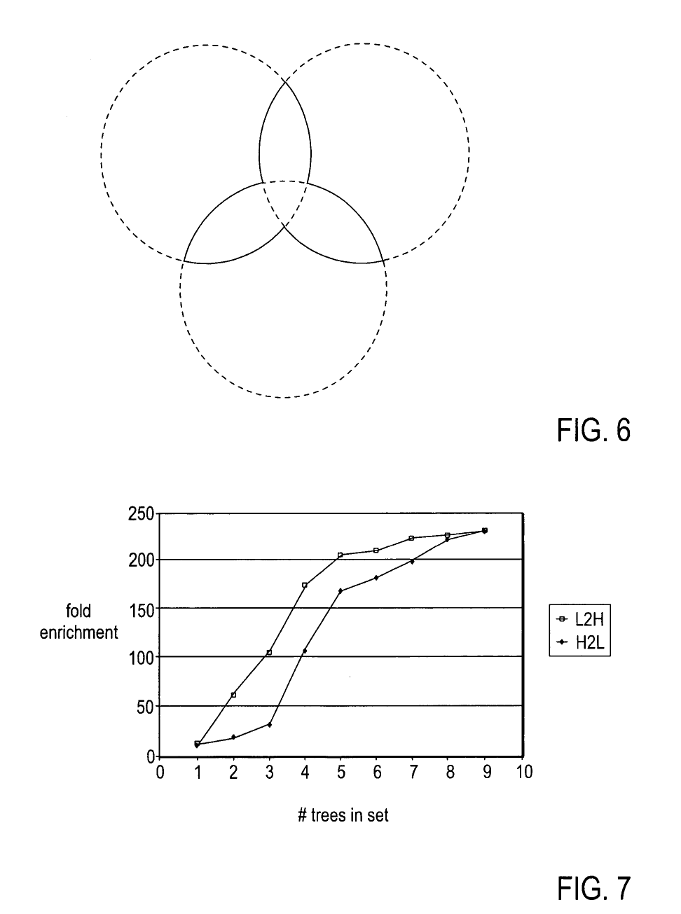

“Unanimous Election” is an extreme form of consensus selection requiring that all individual models in the suite assent to the final solution. “Majority Rule Election” is a related process for group decision-making. It is a means by which solutions are preferentially selected from several models by weighing the input of each of the individual models, and selecting only those solutions that meet the criteria defined by the majority of the participating models. The Majority Rule Election process involves the determination of the Boolean union of intersections of a set of models. It differs from Unanimous Election in that a simple majority of models, requiring at least 3 and preferably an odd number of models to participate, is sufficient to elect a solution (FIG. 6, the area contained in the solid lines). Having an odd number of models in the suite avoids the possibility of a tie in the vote. It is possible to employ the technique with an even number of models, but this may result in indeterminates.

Either process is expected to have a higher, although not equivalent, probability of eliminating false positives from the process, thereby reducing operating cost and throughput requirements, shortening timelines, and increasing the reliability of the process.

The consensus selection methodology has been used previously in alignment problems (Ravi, M., Hopfinger, A. J., Hormann, R. E., Dinan, L. 4D-QSAR Analysis of a Set of Ecdysteroids and a Comparison to CoMFA Modeling. J. Chem. Inf. Comput. Sci. 2001, 41, 1587-1604; Charifson, P. S., Corkery, J. J., Murcko, M. A., Walters, W. P. Consensus Scoring: A Method for Obtaining Improved Hit Rates from Docking Databases of Three-Dimensional Structures into Proteins. J. Med. Chem. 1999, 42, 5100-5109) and in deterministic modeling procedures, (e.g., Kastenholz, M. A., Pastor, M., Cruciani, G., Haaksma, E. E. J., Fox, T. GRID/CPCA: A New Computational Tool to Design Selective Ligands. J. Med. Chem. 2000, 43, 3033-3044) but has not, until recently, been associated with probabilistic modeling methods such as RP. RP has been combined with k-Nearest Neighbor approaches (Miller, D. W. Results of a New Classification Algorithm Combining K Nearest Neighbors and Recursive Partitioning. J. Chem. Inf. Comput. Sci. 2001, 41, 168-175), and chemical class-based homogeneity approaches (Miller, D. W. A Chemical Class-Based Approach to Predictive Model Generation. J. Chem. Inf. Comput. Sci. 2003, 43, 568-578) to similarly improve the performance of the method. . A Random Forest-based RP. technique was recently shown to be effective in modeling CYP450 activity (Ekins, S., Berbaum, J., Harrison, R. K. Generation and Validation of Rapid Computational Filters for CYP2D6 and CYP3A4. Drug Metab. Dispos. 2003, 31, 1077-1080).

As noted above, the consensus selection process involves the determination of the Boolean intersection of a set of models (at least 2, in theory unlimited, individually derived models), thereby emphasizing the probabilities of the consensus set, and de-emphasizing the probabilities of the contributors for each of the models excluded from the consensus set, i.e., the dissenting sets. The process has a higher chance of eliminating false positives from the process, thereby reducing operating costs, throughput requirements, and timelines, while increasing the reliability of the process.

As noted above, two or more recursive partitioning trees may be formed from at least some of the compounds in the test library. The same or different sets of compounds may be used to form the different recursive partitioning trees. If the same sets of compounds are used, then the characteristics of the trees may differ in some way. For example, the tree depth and/or the minimum samples per node may be varied to produce different recursive partitioning trees using the same set of compounds. Alternatively, different sets of compounds from a test library may be used to form respectively different recursive partitioning trees.

The Boolean intersection of the results of two or more recursive partitioning trees may be used to form a consensus set. For example, a first set of compounds is identified using a first recursive partitioning tree formed using a first descriptor set, and a second set of compounds is identified using a second recursive partitioning tree formed using a second descriptor set. A consensus model may then identify compounds that are common to both the first and second sets of compounds. A computer may identify the compounds that are common to both the first and second sets automatically. The identified compounds can form the consensus set. As will be shown in more detail below, the number of compounds identified by the consensus model is less than the number of compounds identified by each recursive partitioning tree used to form the consensus model. The number of identified compounds and the false positive rate are reduced, while maintaining a high fold-enrichment.

When picking trees to form a consensus model, it is desirable to select trees of optimal size for a consensus model, rather than for an individual tree model. For example, multiple trees may be generated using each of three different descriptor sets X, Y, and Z by varying model forming parameters such as tree depth and maximum knots. One may determine that three trees respectively generated from descriptor sets X, Y, and Z differentiate the compound data well, and are similar in that they all have tree depths that are close to each other (e.g., between 14-16). The three trees may then be selected for use in the consensus model, even though the optimal tree depth for the trees formed using the descriptor set X (when viewed independently of the other trees formed using other descriptor sets) may be greater than 16. In some embodiments, it is also desirable to select trees that have similar characteristics when forming a model. For example, in some embodiments, the differences between similar tree characteristics such as tree depth, maximum knots, % class correct, fold enrichment, and % false positives may be less 50%, or even less than about 10%.

Embodiments of the invention have a number of advantages. Since the number of identified compounds is reduced using consensus selection, without increasing the false positive rate and without negatively affecting the fold enrichment, the costs associated with discovering potentially useful compounds are reduced. For example, as discussed in further detail below (Table, FIG. 9), consensus model 4 was formed using three recursion trees respectively formed from the C45, BCUT, and RDF descriptor sets. Consensus model 4 identified 70 actives out of 106 retrieved from over about 22,000 compounds, and had a 100% class correct, a 208.15-fold enrichment, and a 33.96% false positive rate. Consensus model 6 had even better results. Consensus model 6 was formed using nine recursion trees. Three trees were formed using C45, three trees were formed using BCUT, and three trees were formed using RDF (varying the maximum knots). Consensus model 6 identified 70 actives out of 74 retrieved from about 22,000 compounds, and had a 100% class correct, a 298.16 fold enrichment, and a 5.41% false positive rate. These favorable results are compared to the individual models in FIG. 8 which each show a greater than 95% false positive rate in each of the individual trees. At present day cost, it may cost between about 10-55 dollars to test a single candidate compound. Embodiments of the invention can reduce the number of compounds tested by thousands or even tens of thousands. Accordingly, the cost savings that can be achieved by embodiments of the invention can be quite substantial.

Functions such as the selection of compounds using a therapeutic or pharmaceutical profile, the creation of the first and second analytical models (i.e., the creation of descriptors or trees, and the optimization and/or selection of models), the application of the analytical model to a test set, the determination of a consensus set, etc., can be performed using a digital computer that executes code embodying these and other functions. The code may be stored on any suitable computer readable media. Examples of computer readable media include magnetic, electronic, or optical disks, tapes, sticks, chips, etc. The code may also be written in any suitable computer programming language including, C, C++, etc. The software modules may be written in a software development environment such as SPL, SQL and/or C2*SDK, the shell (e.g., the C-shell or Korn shell) environment, or the programming language relevant to the particular application program being used.

The digital computer used in embodiments of the invention may be a micro, mini or large frame computer, or combination thereof, using any standard or specialized operating system such as a UNIX, Linux, or Windows™ based operating system. It is understood that the digital computer that is used in embodiments of the invention could be one or more computational apparatuses that may be together or spatially separated from each other, and may operate using any suitable computer code.

Moreover, any suitable computer database may be used to store any data relating to the test library, test set, training set, or analytical models. Preferably, a computer database such as an Oracle™ relational database management system is used to store this information.

### IV. EXAMPLES

Standardized 3D starting geometries were obtained for all compounds using the UNITY “dbtranslate” utility (version 4.3; Tripos Inc., St. Louis, Mo.) in conjunction with the CONCORD program (version 4.06; developed by R. S. Pearlman, A. Rusinko, J. M. Skell, and R. Baducci at the University of Texas, Austin, and distributed by Tripos Inc., St. Louis, Mo.). 229 Descriptors were calculated using Cerius² (version 4.5; Accelrys Inc., San Diego, Calif.; selected from the following categories: 7 Electronic, 14 Information Content, 7 Molecular Shape Analysis, 49 Spatial, 5 Structural, 117 Thermodynamic, and 30 Topological). 72 Descriptors were calculated using Diverse Solutions (version 4.1.0; developed by R. S. Pearlman, and K. M. Smith at the University of Texas, Austin, and distributed by Tripos Inc., St. Louis, Mo.; BCUT descriptors with explicit hydrogens). 606 Descriptors were calculated using DRAGON PRO (version 3.1; developed by R. Todeschini, V. Consonni, A. Mauri, and M. Pavan at the University of Milano—Bicocca, Milano, Italy, and distributed by Talete srl, Milano, Italy; 197 GETAWAY descriptors; 160 3D-MoRSE descriptors; 99 WHIM descriptors; 150 Radial Distribution Function (RDF) descriptors).

A training set was designed taking the following categories of descriptors into consideration: Electronic, Information Content, Molecular Shape Analysis, Spatial, Structural, Thermodynamic, Topological, and BCUT. The molecular weight and logP-related descriptors were expressly not considered for diversity selection. The remaining 251 descriptors were subjected to Multi-Dimensional Scaling (MDS) as implemented in Cerius² (version 4.6). The MDS analysis (Ochiai Similarity Coefficient, Mean/Variance scaling, Maximum Distance of 10.0) yielded 44 dimensions explaining >90% of the total variance. The 44 dimensions were used in Diverse Compound Selection through a D-optimal Design (Euclidian distance metric, Mean/Variance scaling, 75,000 Monte Carlo Steps at 300 K, Monte Carlo Seed of 12,379, termination after 1,000 idle steps, Gaussian alpha of 1.0, bucket size of 21 for the K-d tree, and taking the nearest 7 neighbors into consideration), as implemented in Cerius (version 4.6). Out of a targeted 25,000 compounds, 22,064 compounds could be obtained from then available stock of the following vendors: AsInEx Inc., Moscow, Russia, ChemBridge Inc., San Diego, Calif., and ChemDiv Inc., San Diego, Calif. The training set was subsequently submitted to a proprietary high-throughput screening (HTS) procedure.

A method optimization and evaluation protocol was written that systematically varied the RP conditions implemented in Cerius² (version 4.6). The terms-used herein are defined in the Cerius² software manual. The following conditions were considered:
Weighting by:
   Classes
   i.e., each class is considered of equal importance to the model rather than each compound
Splitting Method:
   Twoing
   i.e., the formalism that determines how groups are divided or partitioned into statistically distinct nodes or subgroups
Pruning:
   Moderate
   i.e., the procedure that determines the appropriate statistically significant tree depth for each node
Minimum Number of Samples per Node:
   70 (see HTS Results below)
   i.e., a node or subgroup cannot contain fewer than this number of compounds from the training set
Maximum Number of Knots per Split:
   systematically varied using prime numbers starting at 2 and terminating at 199
   i.e., the maximum number of ways a descriptor range may be divided before statistical relevance is determined
Maximum Tree Depth:
   5 through 21
   i.e., the maximum number of splits that may occur before the partitioning process terminates

The 335 combined Cerius², BCUT and RDF descriptors of the training set were submitted to various data reduction techniques. Principal Components Analysis (PCA) was performed using Cerius² version 4.6 explaining 90% of the total variance, and yielding 38 principal components. Factor Analysis (FA) was performed using Cerius² version 4.6 using Multiple Correlation as the Factor Extraction Method, Varimax as the Factor Rotation algorithm, and Auto determination of the Number of Factors. The procedure yielded 98 factors explaining 99.9% of the total variance. Multi-Dimensional Scaling (MDS) was performed using Cerius² version 4.6 using the Ochiai Similarity Coefficient, 70 Dimensions, Mean/Variance scaling, and a Maximum Distance of 10.0, and explained 96.4% of the total variance.

A. Results and Discussion

1. HTS Results.

The HTS procedure yielded 115 hits (in excess of a threshold of 60% activity, which corresponded to about 4×the S. D. of the assay), which were subsequently tested in a concentration-response experiment. 101 of the 115 hits were confirmed to have significant and demonstrable activity by the concentration-response experiment, resulting in a 0.458% true positive/overall hit rate, and a 0.063% false positive rate. 70 compounds with an EC₅₀ value of <10 μM were considered “highly active” and were assigned an activity class of 1 (0.317%). The remaining 21,994 compounds were considered “weakly active” or “inactive”, and were assigned an activity class of 0.

2. Consensus Selection

Previously, it was believed that the number of models to be considered for ensemble methods was more an economical than a scientific decision. To assess this, the fold enrichment and the class correct were tracked in one experiment using the Factor Analysis descriptors, and increased the number of trees in the ensemble (FIG. 7). It became apparent that there exists an asymptotic relationship between the number of trees in the ensemble and the fold enrichment. The class correct was maintained at 100% throughout. In this particular case, adding up to 5 trees to the ensemble provided considerable advantages, but adding more than 5 trees to the ensemble did not increase the performance to a great extent. Therefore, there is an upper limit to both the economic and scientific benefit of adding trees to an ensemble. This behavior is expected, since the best possible model will have 100% sensitivity and 100% specificity, i.e., it will correctly identify all actives, and only the actives. For this particular model that means that the theoretical maximum fold enrichment is restricted to 22,064 / 70 =315.2-fold enrichment. FIG. 7 suggests that the theoretical maximum fold enrichment cannot be achieved with this ensemble of RP trees.

Furthermore, it was found that adding models to an ensemble can have distinct effects depending on the sequence (chronology) in which they were added. FIG. 7 illustrates the hysteresis that occurs when trees are added starting at the logically defined lowest knot limit (van Rhee, A. M., Stocker, J., Printzenhoff, D., Creech, C., Wagoner, P. K., Spear, K. L. Retrospective Analysis of an Experimental High-Throughput Screening Data Set by Recursive Partitioning. J. Combi. Chem. 2001, 3, 267-277.) (low to high, identified as L2H) and when the same set of trees is added in reverse order (high to low, identified as H2L). Although the endpoint of the 9-member ensemble is exactly the same regardless of the direction followed, the 3-member L2H ensemble (106-fold enrichment) has a distinct advantage over the 3-member H2L ensemble (33-fold enrichment). There are no substantial differences between any of the 9 trees in the ensemble, neither in class correct (all 100%) nor in fold enrichment (all between 11.6 and 14.2). Although FIG. 7 is specific to the optimization traces obtained with the Factor Analysis descriptors, similar trends were observed with the C45 and the BCUT descriptors, and the phenomenon is presumably general in nature. A possible explanation lies in the particular way RP is implemented in Cerius²: a maximum knot size needs to be defined for each tree. Consequently, adding a finer grid to a coarse base model, i.e., L2H, provides a bigger advantage than adding a coarser grid to a fine base model, i.e., H2L. Other implementations of RP, such as “rpart” in S-Plus (version 6.0; Insightful, Seattle, Wash.) automatically calculate the optimal split value, and are therefore not subject to hysteresis.

One of the more pervasive problems in cheminformatics is the abundance of ways to describe “chemical diversity space”.It is desirable to select relevant metrics, and preferably metrics with a high discriminatory power. Descriptors from various sources were evaluated, among them ISIS keys (ISIS/Host 5.0; MDL, San Leandro, Calif.), UNITY 2D FingerPrints, Diverse Solutions' BCUT metrics, Cerius² descriptors including E-state keys (Kier, L. B., Hall, L. H. Molecular Structure Description—The Electrotopological State, Academic Press, San Diego, (1999)), GETAWAY (Consonni, V., Todeschini, R., Pavan, M. Structure/Response Correlations and Similarity/Diversity Analysis by GETAWAY Descriptors. 1. Theory of the Novel 3D Molecular Descriptors. J. Chem. Inf. Comp. Sci. 2002, 42, 682-692; and Consonni, V., Todeschini, R., Pavan, M., Gramatica, P. Structure/Response Correlations and Similarity/Diversity Analysis by GETAWAY Descriptors. 2. Application of the Novel 3D Molecular Descriptors to QSAR/QSPR Studies. J. Chem. Inf. Comp. Sci. 2002, 42, 693-705), 3D-MoRSE, (Schuur, J., Selzer, P., Gasteiger, J. The Coding of the Three-Dimensional Structure of Molecules by Molecular Transforms and Its Application to Structure-Spectra Correlations and Studies of Biological Activity. J. Chem. Inf. Comp. Sci. 1996, 36, 334-344; and Gasteiger, J., Sadowski, J., Schuur, J., Selzer, P. Chemical Information in 3D Space. J. Chem. Inf. Comp. Sci. 1996, 36, 1030-1037), Radial Distribution Function (Hemmer, M. C., Steinhauer, V., Gasteiger, J. Deriving the 3D structure of organic molecules from their infrared spectra. Vibrat. Spect. 1999, 19, 151-164; and Hemmer, M. C., Gasteiger, J. Prediction of three-dimensional molecular structures using information from infrared spectra. Anal. Chim. Acta 2000, 420, 145-154) and WHIM (Todeschini, R., Lasagni, M., Marengo, M. New Molecular Descriptors for 2D- and 3D-structures. Theory. J. Chemometrics 1994, 8, 263-273; Todeschini, R., Gramatica, P. 3D-Modelling and Prediction by WHIM Descriptors. Part 5. Theory Development and Chemical Meaning of WHIM Descriptors. Quant. Struct. -Act. Relat. 1997, 16, 113-119; and Todeschini, R., Gramatica, P. 3D-Modelling and Prediction by WHIM descriptors. Part 6. Applications of WHIM descriptors in QSAR Studies. Quant.Struct.-Act.Relat., 1997, 16, 120-125) descriptors. Although complete descriptor sets were calculated, only selected descriptor sets will be discussed herein.

Various techniques were used to try and reduce the dimensionality of the matrix, namely, Principal Component Analysis (PCA), Factor Analysis (FA), and Multi-Dimensional Scaling (MDS), although other techniques such as Fast Random Elimination of Descriptors (FRED) (Waller, C. L., Bradley, M. P. Development and Validation of a Novel Variable Selection Technique with Application to Multidimensional Quantitative Structure-Activity Relationship Studies. J. Chem. Inf. Comput. Sci. 1999, 39, 345-355) and Differential Shannon Entropy (DSE) (Godden, J. W., Bajorath, J. An Information-Theoretic Approach to Descriptor Selection for Database Profiling and QSAR Modeling. QSAR Comb. Sci. 2003, 22, 487-497; Godden, J. W., Bajorath, J. Differential Shannon Entropy as a Sensitive Measure of Differences in Database Variability of Molecular Descriptors. J. Chem. Inf. Comput. Sci. 2001, 41, 1060-1066) may also be applicable. While dimensionality reduction, and more importantly, orthogonality of the data matrix are essential to well defined parametric models, RP by its very design is a dimensionality reduction technique as well as a model building technique that is not sensitive to orthogonality or statistical distribution (Breiman, L., Friedman, J. H., Olshen, R. A., Stone, C. J. Classification and Regression Trees, Wadsworth, Boca Raton, (1984)). The advantage of reducing the dimensionality of the matrix prior to building RP models lies largely in the reduced time and computing resource requirements, and partly in aesthetics.

The table shown in FIG. 9 describes various ways to derive models using consensus selection by RP trees. Consensus models 1, 2, and 3 were constructed for individual descriptor sets as described previously in U.S. patent application Ser. Nos. 60/442,449 filed on Jan. 24, 2003, and Ser. No. 10/754,484, filed on Jan. 9, 2004, which are herein incorporated by reference. Consensus model 4 was constructed using one model each from 3 selected descriptor sets. Consensus model 5 was constructed after applying the Factor Analysis data reduction technique to the matrix. Consensus model 6 is the consensus model resulting from the consensus selection of the 3 previously defined consensus models 1, 2, and 3.

Conceptually, consensus models 1, 2, and 3 are easiest to understand. Each consensus model is built from a single, presumably internally consistent, data matrix. Models were created for all other available metrics, but could not match the existing models based on either % class correct, fold enrichment, or both. All three models retain excellent sensitivity (100%) and achieve very good specificity (98.84%, 97.37%, and 96.26%, respectively). In the training set, the Cerius² descriptors outperform the BCUT metrics by about 2-fold, and the RDF descriptors by less than 3-fold.

Consensus model 4 was created to specifically address an observation reported in a previous publication, (van Rhee, A. M. Use of Recursion Forests in the Sequential Screening Process: Consensus Selection by Multiple Recursion Trees. J. Chem. Inf. Comp. Sci. 2003, 43, 941-948) namely pairing independently derived descriptor bases has been shown to increase the false negative rate under some conditions. It was speculated that this may have resulted from an inadequate or incomplete parametrization of chemical diversity space by the individual descriptor bases. There are no autologous descriptors between the C45, the BCUT, and the RDF descriptor sets, since they each derive their information from a completely different theoretic basis. There is no equivalent for the C45/Kappa-3 descriptor in either BCUT or RDF space, nor is there an equivalent for BCUT/Burden numbers in either C45 or RDF space, or an equivalent for the RDF/120m metric in either C45 or BCUT space. Indeed, the model is predicted to outperform the consensus models created from homogeneous metrics. A 100% sensitivity is retained, and the fold enrichment is boosted by at least a factor of 3 over the best model. Therefore, the observation made previously does not seem to be generally applicable, and may have been a result of the interaction between the specific underlying data structure and the chemical diversity space metric.

Although no autologous descriptors are present in the C45, BCUT, and RDF metric sets, co-linearities or interactions may still exist between the various descriptors. Consensus model 5 was created to find out whether it might be advantageous to reduce or eliminate such interactions prior to the RP model building phase. Of the three data reduction techniques tested, FA yielded the best performing individual models (the table in FIG. 8), possibly because of the greater coverage of the total variance (99.9%) relative to MDS (96.4% ) or PCA (90%). The individual FA RP trees even appear to have a slight advantage over the trees derived from the respective separate metrics, which is apparent in the % of false positives observed. The consensus model obtained with the FA metrics (model no. 5), however, performed less well than the consensus model obtained with the raw metrics (model no. 4), and this appears to be mostly due to a significant increase in the % of false positives observed (FIG. 9). The dimensionality reduction techniques used retain all variables in a compressed form and favor those variables with the highest variance, whether relevant to the subsequent analysis or not. The effect is that variables with small variance that may carry high discriminatory power are de-emphasized prior to assessing relevance. The resulting models may therefore be biased against the most discriminating metrics, which could result in higher false positive rates, and possibly higher false negative rates, although the latter was not observed. Alternatively, because the dimensionality reduction techniques used here minimize interactions between descriptors, the process may be eliminating interactions highly relevant to the active compounds, but less relevant to the inactive compounds, thereby reducing specificity. Reduced specificity is equivalent to an increase in the number of false positives observed.

Having established that the 9-member FA forest (FIG. 7) only marginally outperforms the 3-member raw metric ensemble (FIG. 9, No. 4), and knowing the upper limit to the fold enrichment (315.2-fold; see above), it was investigated whether an extended raw metric ensemble could achieve better performance than previously established models. Consensus model 6 describes this ensemble. Consensus model 6 has a 100% sensitivity, a 99.98% specificity, and a 99.98% concordance, resulting in a 298-fold enrichment. The model predicted only four compounds more than the known 70 actives to be active. These four compounds were resubmitted to the concentration-response experiment, and established that 2 compounds had an EC₅₀ of <10 μM, 1 compound had an EC₅₀ of about 20 μM, and the remaining compound showed no activity. Thus, the original screen missed at least 2 false negative compounds, and the current model correctly identified 72/74 compounds, which translates into a 97.3% hit rate, and reduces the maximum fold enrichment to 306.4.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed.

All patent applications, patents, and publications above are herein incorporated by reference. None is admitted to be prior art.

1. A method for screening compounds for biological activity comprising:
a) selecting a test library of compounds;
b) forming a first analytical model using a first recursive partitioning process that uses a first descriptor set, a digital computer, and at least some of the compounds in the test library of compounds;
c) forming a second analytical model using a second recursive partitioning process that uses a second descriptor set, the digital computer, and at least some of the compounds in the test library of compounds; and
d) determining a consensus compound set using at least the first analytical model and the second analytical model,
wherein the first descriptor set and the second descriptor set have different groups of descriptors.

2. The method of claim 1 further comprising:
forming a third analytical model using a third recursive partitioning process using a third descriptor set, the digital computer, and at least some of the compounds in the test library of compounds,
wherein determining the consensus compound set further includes using the third analytical model in addition to the first analytical model and the second analytical model, and
wherein the third descriptor set has a different group of descriptors than both the first and the second descriptor sets, wherein the false positive rate of the consensus compound set is less than about 50%.

3. The method of claim 1 wherein the compounds that are used to form the first and second analytical models are the same.

4. The method of claim 1 wherein the compounds that are used to form the first and the second analytical models are different.

5. The method of claim 1 wherein the compounds that are used to form the first and the second analytical models are the same and constitute a training set of the library of compounds.

6. The method of claim 1 wherein the test library of compounds comprise ion channel modulators.

7. The method of claim 1 wherein d) is performed by the digital computer.

8. The method of claim 1 wherein determining the consensus compound set includes identifying compounds that are predicted to be active by both the first analytical model and the second analytical model.

9. A computer readable medium comprising:
a) code for selecting a test library of compounds;
b) code for forming a first analytical model using a first recursive partitioning process that uses a first descriptor set, a digital computer, and at least some of the compounds in the test library of compounds;
c) code for forming a second analytical model using a second recursive partitioning process that uses a second descriptor set, the digital computer, and at least some of the compounds in the test library of compounds; and
d) code for determining a consensus compound set using at least the first analytical model and the second analytical model,
wherein the first descriptor set and the second descriptor set have different groups of descriptors.

10. The computer readable medium of claim 9 further comprising:
code for forming a third analytical model using a third recursive partitioning process using a third descriptor set, the digital computer, and at least some of the compounds in the test library of compounds,
wherein determining the consensus compound set further includes using the third analytical model in addition to the first analytical model and the second analytical model, and
wherein the third descriptor set has a different group of descriptors than both the first and the second descriptor sets.

11. The computer readable medium of claim 9 wherein the compounds that are used to form the first and second and third analytical models are the same.

12. The computer readable medium of claim 9 wherein the compounds that are used to form the first and the second and the third analytical models are different.

13. The computer readable medium of claim 9 wherein the compounds that are used to form the first and the second and the third analytical models are the same and constitute a training set of the library of compounds.

14. The computer readable medium of claim 9 wherein the test library of compounds comprise ion channel modulators.

15. The computer readable medium of claim 9 wherein the digital computer is embodied by two or more computational apparatuses.

16. The computer readable medium of claim 9 wherein determining the consensus compound set includes identifying compounds that are predicted to be active by both the first analytical model and the second analytical model.

17. The computer readable medium of claim 9 wherein the first and second analytical models are two of more than one hundred analytical models that are used to form the consensus compound set.

18. The computer readable medium of claim 9 wherein the first and second analytical models are two of more than one thousand analytical models that are used to form the consensus compound set.

19. The computer readable medium of claim 9 wherein a unanimous election process is used to determine the consensus compound set.

20. The computer readable medium of claim 9 wherein a majority rule election process is used to determine the consensus compound set.

21. A method for screening compounds for biological activity comprising:
a) selecting a test library of compounds;
b) forming a first analytical model using a first recursive partitioning process that uses a first descriptor set, a digital computer, and at least some of the compounds in the test library of compounds;
c) forming a second analytical model using a second recursive partitioning process that uses a second descriptor set, the digital computer, and at least some of the compounds in the test library of compounds;
d) forming a third analytical model using a third recursive partitioning process that uses a third descriptor set, the digital computer, and at least some of the compounds in the test library of compounds; and
e) determining a consensus compound set using at least the first, second, and third analytical models,
wherein the first, second, and third descriptor sets have different groups of descriptors.

22. The method of claim 21 further comprising:
selecting the first, second, and third analytical models so that characteristics of the models are similar.

23. A computer readable medium comprising:
a) code for selecting a test library of compounds;
b) code for forming a first analytical model using a first recursive partitioning process that uses a first descriptor set, a digital computer, and at least some of the compounds in the test library of compounds;
c) code for forming a second analytical model using a second recursive partitioning process that uses a second descriptor set, the digital computer, and at least some of the compounds in the test library of compounds;
d) code for forming a third analytical model using a third recursive partitioning process that uses a third descriptor set, the digital computer, and at least some of the compounds in the test library of compounds; and
e) code for determining a consensus compound set using at least the first, second, and third analytical models, wherein the first, second, and third descriptor sets have different groups of descriptors.

24. The computer readable medium of claim 23 further comprising:
code for selecting the first, second, and third analytical models so that characteristics of the models are similar.
